(19) United States
(12) Patent Application Publication
(10) Pub. No.: US 2002/0189027 A1
(43) Pub. Date: Dec. 19, 2002

(54) Composition and methods for lanthionizing keratin fibers using at least one organic nucleophile and at least one hydroxide ion generator

(76) Inventors: David W. Cannell, Plainfield, NJ (US); Hitendra Mathur, Woodbridge, NJ (US); Nghi Van Nguyen, Edison, NJ (US)

Correspondence Address: FINNEGAN, HENDERSON, FARABOW, GARRETT &, DUNNER LLP, 1300 I STREET, NW, WASHINGTON, DC, 20005, US

(21) Appl. No.: 09/838,197

(22) Filed: Apr. 20, 2001

(51) Int. Cl. A61K 7/13

(52) U.S. Cl. 8/405; 8/406; 8/415

(57) ABSTRACT

Compositions for lanthionizing keratin fibers comprising at least one organic nucleophile and at least one hydroxide ion generator, wherein the at least one organic nucleophile is present in an amount effective to increase the tensile strength of the keratin fibers. Pretreatment compositions for keratin fibers comprising at least one organic nucleophile, wherein the pretreatment composition is applied prior to applying a relaxing composition and further wherein the at least one organic nucleophile is present in an amount effective to increase the tensile strength of the keratin fibers. Methods and multicomponent kits for lanthionizing keratin fibers to achieve relaxation of the keratinous fibers using at least one organic nucleophile.

The present invention relates to compositions for lanthionizing keratin fibers comprising at least one organic nucleophile and at least one hydroxide ion generator, wherein said at least one organic nucleophile is present in an amount effective to increase the tensile strength of the keratin fibers, and methods for using the same. The inventive compositions may result in relaxed or straightened hair with improved mechanical properties. The invention also provides pretreatment compositions for lanthionizing keratin fibers comprising at least one organic nucleophile, and methods for using the same.

Straightening or relaxing the curls of very curly hair may increase the manageability and ease of styling of such hair. In today's market, there is an increasing demand for the hair care products referred to as “hair relaxers,” which can relax or straighten naturally curly or kinky hair. Hair relaxers may either be applied in a hair salon by a professional or in the home by the individual consumer.

Hair fiber, a keratinous material, comprises proteins (polypeptides). Many of the polypeptides in hair fibers are bonded together or cross-linked with disulfide bonds (—S—S—). A disulfide bond may be formed from the reaction of two sulfhydryl groups (—SH), one on each of two cysteine residues, which results in the formation of a cystine residue. A cystine residue comprises a cross-link of the formula —CH₂—S—S—CH₂-between 2 polypeptides. While there are other types of bonds which occur between the polypeptides in hair fibers, such as ionic (salt) bonds, the permanent curling or the shape of the hair is essentially dependent on the disulfide bonds of cystine residues.

As a result, relaxing or straightening of hair can be achieved by disrupting the disulfide bonds of the hair fibers with an alkaline agent or a reducing agent. The chemical disruption of disulfide bonds by an alkaline agent is usually combined with mechanical straightening of the hair, such as combing, and straightening generally occurs due to changes of the relative positions of opposite polypeptide chains within the hair fiber. The reaction is generally terminated by rinsing and/or the application of a neutralizing composition.

The reaction with the alkaline agent is normally initiated by hydroxide ions. Specifically, hydroxide ions initiate a reaction in which a cystine cross-link (—CH₂—S—S—CH₂—) is broken and a lanthionine cross-link (—CH₂—S—CH₂—) is formed. The lanthionine cross-link is shorter than a cystine cross-link by one sulfur atom, and thus the net effect of the reaction is to reduce the distance between polypeptides. Amino acid analysis indicates that from 25 mole % to 40 mole % of cystine residues are converted to lanthionine residues.

Not to be limited by theory, there are two reaction sequences that are predominantly used in the art to explain the disruption of the disulfide bonds in hair fibers by hydroxide ions. As previously mentioned, both of these reaction sequences result in lanthionine residue formation. Consequently, the term “lanthionizing” is used when one skilled in the art refers to the relaxing or straightening of keratin fibers by hydroxide ions.

One reaction sequence comprises at least one bimolecular nucleophilic substitution reaction wherein an available hydroxide ion directly attacks the disulfide linkage of a cystine residue. The result is the formation of lanthionine residues and HOS⁻. See Zviak, C., The Science of Hair Care, 185-186 (1986). The second reaction sequence comprises at least one β-elimination reaction initiated by the nucleophilic attack of an available hydroxide ion on a hydrogen atom bonded to a carbon atom that is in the β-position with respect to the disulfide bond of a cystine residue. Id. The result is the formation of a dehydroalanine residue which comprises a reactive double bond (═CH₂). The double bond of the dehydroalanine residue can then react with the thiol group of a cysteine residue to form a lanthionine residue.

Most frequently, commercial relaxing compositions are in the form of gels or emulsions and contain varying proportions of strong water-soluble bases, such as sodium hydroxide (NaOH), or of compositions that contain slightly-soluble metal hydroxides, such as calcium hydroxide (Ca(OH)₂), which can be converted in situ to soluble bases, such as guanidine hydroxide.

Traditionally, the two main hair relaxers used in the hair care industry for generating hydroxide ions are referred to as “lye” (lye=sodium hydroxide) relaxers and “no lye” relaxers. The “lye” relaxers generally comprise sodium hydroxide in a concentration ranging from 1.5% to 2.5% by weight relative to the total weight of the composition (0.38M -0.63 M) depending on the carrier used, the condition of the hair fibers and the desired length of time for the relaxation process. Sodium hydroxide may be extremely effective in straightening the hair but may result in a reduction of the strength of the hair fibers and, in some cases, partial or total loss of hair due to hair fiber breakage.

Some manufacturers market lithium and potassium hydroxide relaxers as “no lye” but, while they technically do not contain lye, these relaxers may still rely on the soluble hydroxides of inorganic metals, such as potassium and lithium. For example, a curing method for permanent hair straightening using thioglycolic acid, dithioglycolic acid, and potassium hydroxide is known. See Ogawa, S. et al., J. Cosmet. Sci., 51, 379-399 (2000). This method comprises three steps: (1) reduction using thioglycolic acid (3% to 9%), dithioglycolic acid (up to 4%), potassium hydroxide, EDTA and monoethanolamine; (2) heat treatment, followed by (3) oxidation of the hair.

Other “no lye” relaxers may use hydroxide ions obtained from, for example, a slightly soluble source such as Ca(OH)₂. For example, the slightly soluble Ca(OH)₂ is mixed with guanidine carbonate to form a soluble but unstable source of hydroxide ions, guanidine hydroxide, and the insoluble calcium carbonate (CaCO₃). The reaction is driven to completion by the precipitation of CaCO₃ and is in effect substituting one insoluble calcium salt for a slightly soluble calcium salt.

Other relaxers may include compositions comprising at least one multivalent metal hydroxide and at least one complexing agent, which is effective for dissociating the at least one multivalent metal hydroxide in sufficient quantities to effect lanthionization of the keratin fibers. Also known are compositions comprising at least one multivalent metal hydroxide and at least one ion exchange resin, which is effective for dissociating the at least one multivalent metal hydroxide in sufficient quantities to effect lanthionization of the keratin fibers. Further, compositions for lanthionization of keratin fibers comprising at least one hydroxide compound and at least one activating agent chosen from cysteine and derivatives thereof are known.

The mechanical properties of hair that has been lanthionized using hydroxide ion generating compositions demonstrate that, while the hair may not be significantly weaker due to the reduction in space between polypeptides (and in fact may have a high yield force), the hair may have a lower elongation before breaking. This “brittleness” of high yield force coupled with low elongation and inherently weaker points (where the hair had natural twists) can lead to breakage during grooming.

Thus, there is still a need for compositions and methods to relax keratin fibers which preserve the relaxing efficiency of hydroxide ion generating compositions in lanthionizing hair, while also providing at least one desired mechanical property to the lanthionized hair.

The present invention, in one aspect, provides a composition for lanthionizing keratin fibers comprising at least one organic nucleophile and at least one hydroxide ion generator, wherein the at least one organic nucleophile is present in an amount effective to increase the tensile strength of the keratin fibers ranging from greater than 0.1% but less than 3% by weight relative to the total weight of the composition, and with the proviso that if the at least one nucleophile is chosen from cysteine and derivatives thereof, then the at least one nucleophile is present in amount greater than 1.5% but less than 3% by weight relative to the total weight of the composition. As used herein, “at least one” means one or more and thus includes individual components as well as mixtureslcombinations. According to the present invention, the keratin fibers are chosen from human keratin fibers, such as hair, eyelashes, and eyebrows. In one embodiment, the keratin fibers are hair.

In another aspect of the invention, the present invention provides a pretreatment composition for keratin fibers comprising at least one organic nucleophile, wherein the pretreatment composition is applied to the keratin fibers prior to applying a relaxing composition, wherein the at least one organic nucleophile is present in an amount effective to increase the tensile strength of the keratin fibers. As used herein, a “relaxing composition” means a composition comprising at least one hydroxide ion generator in an amount sufficient to effect lanthionization of keratin fibers.

In yet another aspect of the invention, the present invention provides a method for lanthionizing keratin fibers to achieve relaxation of the keratin fibers comprising applying to keratin fibers a composition comprising (i) at least one organic nucleophile and (ii) at least one hydroxide ion generator for a sufficient period of time to lanthionize the keratin fibers. The lanthionization is terminated when a desired level of relaxation of the keratin fibers has been reached. The at least one organic nucleophile is present in an amount effective to increase the tensile strength of the keratin fibers ranging from greater than 0.1% but less than 3% by weight relative to the total weight of the composition.

The present invention is also drawn to a method for lanthionizing keratin fibers to achieve relaxation of the keratin fibers comprising applying a pretreatment composition comprising at least one organic nucleophile. The application of the pretreatment composition is followed by application of a relaxing composition for a sufficient period of time to lanthionize the keratin fibers. The lanthionization is terminated when a desired level of relaxation of the keratin fibers has been reached. The at least one organic nucleophile is present in an amount effective to increase the tensile strength of the keratin fibers.

Further, the invention also provides for a multicomponent kit for lanthionizing keratin fibers, wherein the kit comprises at least two components. A first component of the kit contains at least one organic nucleophile, and a second component contains at least one hydroxide ion generator.

It is to be understood that both the foregoing general description and the following detailed description are exemplary and explanatory only and are not restrictive of the invention as claimed.

As described above, the lanthionization of keratin fibers is driven by the release of hydroxide ions, which disrupt the disulfide bond of cystine and allow the formation of lanthionine cross-links. These lanthionine cross-links are believed to make the hair more brittle.

Using amino acid analysis, the inventors have found that, in addition to lanthionine cross-links, other, different, cross-links are formed in treated hair. This suggests either that the reaction rate for the formation of lanthionine cross-links is slow enough to allow competing reactions, orthat the movement of polypeptides through the “smoothing” of the hair separates the potentially reactive species, thereby allowing different cross-links (i.e., non-lanthionine cross-links) to form.

These non-lanthionine cross-links may be formed from the reaction of an organic nucleophile with the reactive double bond (═CH₂) of a dehydroalanine residue. For example, relaxed hair may contain both lanthionine cross-links and lysinoalanine cross-links (—CH₂—NH—(CH₂)₃—). These lysinoalanine cross-links may be formed by reaction of the side chain of a lysine residue (H₂N—(CH₂)₃—) with a dehydroalanine residue.

Further, an increase in the number of serine residues (which have a -CH₂OH side chain) in treated hair may be observed. Serine residues may be formed from the reaction of water with the double bond of a dehydroalanine residue. Obviously, the formation of a serine residue excludes the formation of a cross-link between polypeptides.

Further, the inventors have discovered that when guanidine carbonate and calcium hydroxide are mixed, and the resultant guanidine hydroxide is used as a hydroxide ion generator in a relaxing composition, β-guanidinoalanine residues (having a —CH₂NH—C(NH)—NH₂ side chain) are formed in the treated hair. These β-guanidinoalanine residues may be formed from the reaction of a guanidine residue with the double bond in dehydroalanine. As in the case of serine residue formation, the formation of β-guanidinoalanine residues also results in the absence of a cross-link between polypeptides. Notably, mechanical properties of hair treated with guanidine hydroxide have been observed to be better than those of hair treated with lye relaxers.

Thus, while not wishing to be limited to theory, the present inventors believe that the reaction of organic nucleophiles (such as those above—e.g., cysteine, water, and guanidine), which competeswith the reaction forming lanthionine cross-links, increases at least one mechanical property of the keratinous fibers, such as tensile strength. Modification of the degree of lanthionine cross-linking using such competing reactions may provide at least one desired hair property while, at the same time, preserving the relaxing efficiency of the relaxing composition. For example, the use of at least one organic nucleophile either in a pretreatment composition or in a relaxing composition may increase the tensile strength of relaxed hair while preserving the relaxing efficiency of the relaxing composition. Further, the present invention may permit a balancing of the relaxing effect and the tensile strength.

While known relaxing compositions may contain organic nucleophiles, the presence of such nucleophiles may not be controlled and/or such nucleophiles may not be present in an amount effective to provide increased tensile strength to the keratin fibers. The controlled use of at least one organic nucleophile may provide improved control and accuracy over the degree of relaxation, and, in any event, may provide a desired level of both control and accuracy. For example, by varying the relative concentration of the at least one organic nucleophile, it may be possible to control the relaxing efficiency of the relaxing composition, i.e., to control the ratio of lanthionine cross-links to non-lanthionine cross-links and/or residues (such as β-guanidinoalanine residues). One of ordinary skill in the art would recognize that varying these relative concentrations is not the only method to affect relative reaction rates and, thus, the relaxing efficiency. Moreover, one skilled in the art would know how to balance the desired properties of the degree of relaxation and tensile strength of the treated hair, because they would know that a very high concentration of at least one organic nucleophile could prevent the relaxation of the hair, in part by competing with lanthionization and in part by consuming the generated hydroxide ions. Specifically, to achieve at least one desired mechanical property, one of ordinary skill in the art would know how to take into consideration various factors, such as type of at least one organic nucleophile, type of relaxing composition, degree of penetration by the at least one organic nucleophile (time and concentration), amount of hydroxide ions generated (time and concentration), hair type, and mode of application.

Thus, it is believed that treatment of keratin fibers with a strong localized concentration of at least one organic nucleophile prior to, or during, lanthionization of the keratin fibers would result in a competing reaction for the reactive double bond of the resultant dehydroalanine residue between the at least one organic nucleophile and the thiol group of a cysteine residue. That is, it is believed that there would be competition between the reaction of the at least one organic nucleophile which would result in the formation of residues (i.e., lack of cross-links) and the formation of lanthionine cross-links. As previously mentioned, the at least one organic nucleophile could be incorporated in a relaxing composition itself, the at least one organic nucleophile could be combined with the relaxing composition prior to application to keratin fibers, and/orthe at least one organic nucleophile could be applied to the keratin fibers prior to the application of a relaxing composition.

As used herein, the phrase “relaxing efficiency” refers to the ability of a relaxing composition to straighten hair. The efficiency, % RE, is measured as described below in the Examples. The preservation of relaxing efficiency is evidenced by the maintenance of the % RE of the relaxing composition when a composition comprising at least one organic nucleophile is used before or simultaneously with a relaxing composition as compared to the use of the same relaxing composition without any prior or simultaneous use of at least one organic nucleophile.

As used herein, the phrase “increased tensile strength” refers to an increase in tensile strength in the keratinous fibers observed when a composition comprising at least one organic nucleophile is used before or with a relaxing composition as compared to the use of the same relaxing composition without any prior or simultaneous use of at least one organic nucleophile. The increase in tensile strength is evidenced by a reduction in the value for % Decrease in Work (W), as measured by the procedure described below in the Examples, i.e., when hairtreated with at least one organic nucleophile has less damage (and increased tensile strength) than hair not treated with at least one organic nucleophile, the value for % Decrease in W is comparatively lower.

As used herein, the phrase “at least one organic nucleophile” refers to any organic nucleophile, whether in a stable or unstable form, which is effective in competing with the lanthionization process to increase the tensile strength of the keratin fibers. For example, the at least one organic nucleophile may be chosen from basic amino acids, amines, alcohols, and mercaptans. Likewise, as used herein, “the at least one organic nucleophile source” refers to any source of at least one organic nucleophile, whether in a stable or unstable form, which is effective in competing with the lanthionization process to increase the tensile strength of the keratin fibers. For example, the at least one organic nucleophile source may be chosen from derivatives of basic amino acids, derivatives of amines, derivatives of alcohols, derivatives of mercaptans and any compounds that react in situ to generate at least one organic nucleophile. Non-limiting example of said derivatives include salts.

As used herein, the phrase “basic amino acid” refers to a natural basic amino acid such as, for example, lysine, arginine, and histidine, their isomeric and racemic forms, as well as synthetic basic amino acids, their isomeric and racemic forms, and derivatives of any of the foregoing. In one embodiment of the present invention, the at least one organic nucleophile is chosen from lysine and arginine, which may be in a non-ionic form (such as lysine) and/or in the form of derivatives thereof, e.g., an ammonium form (such as lysine hydrochloride) and/or a carboxylate form (such as sodium lysinate).

Amines for use according to the invention may be chosen from amines of formula (I) and salts thereof:

N(R)₃ (I)

wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups, wherein R may optionally be substituted with at least one group chosen from —COOR, —COON(R)₂, —OH, —SH, —N(R)₂, and salts of any of the foregoing. Obviously, each R of the at least one group is independently chosen from a hydrogen atom, linear, branched, substituted, and unsubstituted C₁-C₁₀ alkyl groups, and linear, branched, substituted, and unsubstituted C₁-C₁₀ alkenyl groups. Non-limiting examples of salts suitable for the present invention include ammonium salts, alkali metal salts, alkaline earth metal salts, organic acid addition salts and inorganic acid addition salts. For example, a suitable alkali metal salt is the salt of the amine formed when at least one R is chosen from —COOH. In one embodiment of the present invention, the amines are chosen from isopropylamine, monoethanolamine, and aminomethylpropanol.

According to the present invention, the at least one organic nucleophile may be chosen from alcohols of formula (II) and salts thereof:

R—OH (II)

wherein R is chosen from linear, branched, substituted, and unsubstituted C₁-C₁₀ alkyl groups, and linear, branched, substituted, and unsubstituted C₁-C₁₀ alkenyl groups, wherein R may optionally be substituted with at least one group chosen from —COOR′, —COON(R′)₂, —OH, —SH, —N(R′)₂, and salts of any of the foregoing, wherein R′ is chosen from a hydrogen atom, linear, branched, substituted, and unsubstituted C₁-C₁₀ alkyl groups, and linear, branched, substituted, and unsubstituted C₁-C₁₀ alkenyl groups.

Mercaptans for use according to the invention may be chosen from mercaptans of formula (III) and salts thereof:

R—SH (III)

wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups, wherein R may optionally be substituted with at least one group chosen from —COOR, —COON(R)₂, —OH, —SH, —N(R)₂, and salts of any of the foregoing. Non-limiting examples of the salts suitable for the present invention include ammonium salts, alkali metal salts, alkaline earth metal salts, organic acid addition salts and inorganic acid addition salts. For example, a suitable alkali metal salt is the salt of the mercaptan formed when at least one R is chosen from —COOH. One skilled in the art would recognize that, in some embodiments, cysteine may not be an acceptable nucleophile because of its activity as a reducing agent.

In one embodiment, the alkyl groups of R in formulae (I), (II), and (III) may comprise from 1 to 4 carbon atoms. For example, in this embodiment, these groups may be chosen from methyl groups, ethyl groups, and propyl groups. In another embodiment, the alkyl groups of R of formulae (I), (II), and (III) may comprise from 1 to 6 carbon atoms. In yet another embodiment, the alkali metal salts and alkaline earth metal salts may be chosen from sodium salts, potassium salts, and calcium salts. In a further embodiment, the organic acid addition salts and the inorganic acid addition salts may be chosen from salts derived from hydrochloric acid, salts derived from sulphuric acid, salts derived from phosphoric acid, salts derived from acetic acid, salts derived from citric acid, and salts derived from tartaric acid.

In one embodiment, the composition of the invention may further comprise at least one additional organic nucleophile different from the at least one organic nucleophile.

The amount of the at least one organic nucleophile that is effective to increase the tensile strength of the keratin fibers is a function of many parameters. One of ordinary skill in the art would recognize that this amount is a function of at least: the type of organic nucleophile, amount and type of relaxer, hair type, mode of application, and the desired effect (balancing of the levels of tensile strength and relaxing efficiency).

When the inventive composition comprises at least one organic nucleophile and at least one hydroxide ion generator, the at least one organic nucleophile may be present in an amount greater than 0. 1% but less than 3% by weight relative to the total weight of the composition. When included in a pretreatment composition, the at least one organic nucleophile is initially taken up by the hair before treatment with the relaxing composition. One of ordinary skill in the art would recognize that the extent of the penetration of a pretreatment is a function of at least time, concentration, type of organic nucleophile, hair type, and mode of application. But, testing has shown that relaxing compositions can remain effective in straightening the hair even at the highest levels of penetration with a pretreatment of at least one organic nucleophile. In one embodiment, the at least one organic nucleophile may be present in any amount up to 100% by weight relative to the total weight of the pretreatment composition comprising at least one organic nucleophile. In another embodiment, the at least one organic nucleophile is present in an amount ranging from 0.001% to 10.0% by weight relative to the total weight of the pretreatment composition, while in yet another embodiment, it is present in an amount ranging from 0.01% to 2%. In one embodiment, the at least one organic nucleophile is present in amount of 0.2% by weight relative to the total weight of the pretreatment composition.

Whether in the form of a pretreatment composition or a relaxing composition, the inventive compositions may further comprise at least one other constituent, which is conventional in cosmetics, chosen from solvents such as alcohol and water; preservatives; perfumes; UV filters; active haircare agents; plasticizers; anionic, cationic, amphoteric, nonionic, and zwitterionic surfactants; hair conditioning agents such as silicone fluids, fatty esters, fatty alcohol, fatty chain hydrocarbons, emollients, lubricants, and penetrants such as lanolin compounds, protein hydrolysates, and other protein derivatives; anionic, cationic, amphoteric, nonionic, and zwitterionic polymers; dyes; tints; bleaches; reducing agents; pH adjusting agents; sunscreens; and thickening agents.

The at least one hydroxide ion generator may be chosen from those compositions that produce hydroxide ions appropriate for the lanthionization of hair.

As used herein, “hydroxide ion generator” refers to both compounds and compositions that generate hydroxide ions, and compounds and compositions that comprise hydroxide ions. For example, in one embodiment, the relaxing composition comprises at least one hydroxide ion generatorwhich generates hydroxide ions in situ. Hydroxide ion generators may, for example, be chosen from traditional “lye” and “no lye” hair relaxer compositions and other soluble or slightly soluble hydroxide ion sources.

Further, the at least one hydroxide ion generator may be used in combination with at least one agent, different from the at least one organic nucleophile, chosen from chelating agents, sequestering agents, and salts thereof. Non-limiting examples of the at least one agent include acids and derivatives thereof comprising at least one electron withdrawing moiety in the a-position with respect to the acid functional group. These acids may, for example, be chosen from carboxylic acids, thiocarboxylic acids, phosphoric acids, and phosphonic acids. The electron withdrawing moiety may, for example, be chosen from groups comprising at least one electron withdrawing atom, such as nitrogen, oxygen and sulfur, and derivatives thereof. Other non-limiting examples of the at least one agent include chelating agents, sequestering agents, and salts thereof described in co-pending U.S. patent application Ser. No. 09/516,942, the disclosure of which is incorporated herein by reference. Thus, the inventive compositions, whether in the form of a pretreatment composition or a relaxing composition, may further comprise at least one agent chosen from chelating agents, sequestering agents and salts of any of the foregoing.

The present invention is also drawn to methods for lanthionizing keratin fibers to achieve relaxation of the keratin fibers. One method of the present invention comprises applying to the keratin fibers a pretreatment composition comprising at least one organic nucleophile as previously described, wherein the at least one organic nucleophile is present in an amount effective to increase the tensile strength of the hair ranging from greater than 0. 1% but less than 3% by weight relative to the total weight of the composition. A relaxing composition is then applied to the keratin fibers for a sufficient period of time to lanthionize the keratin fibers. The lanthionization is terminated when the desired level of relaxation of the keratin fibers has been reached.

A second method of the present invention comprises applying to the keratin fibers for a sufficient period of time to lanthionize the keratin fibers a relaxing composition further comprising at least one organic nucleophile as previously described. The at least one organic nucleophile is present in an amount effective to increase the tensile strength of said keratin fibers. The lanthionization is terminated when a desired level of relaxation of said keratin fibers has been reached. The lanthionization is terminated when the desired level of relaxation of the keratin fibers has been reached.

The compositions of the present invention may also be provided as a one part composition comprising at least one hydroxide ion generator and at least one organic nucleophile or in the form of a multicomponent kit. The multicomponent kit for lanthionizing keratin fibers comprises at least two separate components. A first component of the kit contains at least one composition comprising at least one organic nucleophile. This first component may be in a form chosen from emulsions, solutions, suspensions, gels, creams, and pastes. A second component of the kit contains at least one composition for generating hydroxide ions. This second component may also be in a form chosen from emulsions, solutions, suspensions, gels, creams, and pastes.

The skilled artisan, based on the stability of the composition and the application envisaged, will be able to determine how the composition and/or multicomponent kit should be stored and/or combined.

In one embodiment of the multicomponent kit, the first component is a pre-treatment composition that is applied to the hair prior to the application of the second component. A pretreatment composition may provide a strong localized concentration of organic nucleophiles prior to the introduction of hydroxide ions. In another embodiment, the first component is to be combined with the second component before application. In yet another embodiment, one of the components of the kit will contain enough water or other ionizing solvent to ensure that, upon mixing, enough of the generated hydroxide ions remains in solution to effect lanthionization of keratin fibers.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

### EXAMPLES

### Example 1

### Tensile Strength of Hair Treated with a Relaxing Composition Further Comprising an Organic Nucleophile

The effect of lysinate (an organic nucleophile) in a relaxing composition on relaxed hair was studied by adding various amounts of sodium lysinate to a commercially available lye-containing relaxing composition. Sodium lysinate was added such that the final mixtures contained 2.50% by weight of available generated hydroxide ions and lysinate concentrations as reported in Table 1.

A total of 100 naturally kinky hair fibers were equilibrated in water for 24 hours and then pulled to a 3% extension by a Dia-Stron Mechanical Tensile Tester. The work to extend hair to 3% was recorded as Wᵢ. The same hair fibers were allowed to equilibrate in water for 24 hours and were then treated with one of each of the various relaxing mixtures for20 minutes. Afterthorough rinsing, theworkto extend the relaxed hair to 3% was again recorded, and was recorded as Wf. The % Decrease in Work at 3% extension (% Decrease in W) was calculated according to the following equation:

% Decrease in W=(W_{f}/Wᵢ)×100%

The results are shown in Table 1.

TABLE 1

| % Lysinate | pH of Final Mixture | % Decrease in W |
|---|---|---|
| 0 | 13.96 | 44.88 |
| 0.1 | 14.00 | 46.00 |
| 0.5 | 13.99 | 40.20 |
| 3.0 | 13.99 | 52.40 |
| 10.0 | 14.00 | 61.40 |

The results in Table 1 show that the tensile strength of the treated hair increased (as reflected by the lower % Decrease in W) when the concentration of lysinate was greater than 0.1% but less than 3.0%. In other words, for the aforementioned concentration range, the tensile strength of hair treated with a relaxing composition further comprising at least one organic nucleophile (sodium lysinate) was higher than that of hair treated with a relaxing composition alone (ie. not comprising at least one organic nucleophile).

### Example 2

### Relaxing Efficiency of a Relaxing Composition Further Comprising an Organic Nucleophile

The effect of sodium lysinate in a relaxing composition on relaxed hair was studied by adding various amounts of sodium lysinate to a commercially available lye-containing relaxing composition. Sodium lysinate was added such that the final mixtures contained 2.50% by weight of available generated hydroxide ions and lysinate concentrations as reported in Table 2. After mixing for 30 seconds, each final mixture was applied to a naturally kinky hair swatch that was stretched and taped in a straight configuration. The final mixture was worked into the hair swatch for 5 minutes. The treated hair swatch was allowed to stand at ambient temperature for another 15 minutes. The hair swatch was rinsed and shampooed and then placed in a humidity chamber at 90% Relative Humidity for 24 hours. The % Relaxing Efficiency (% RE) is defined as:

% RE=(L_{f}/Lᵢ)×100%

where

L_{f}=Length of the relaxed hair after 24 hours at 90% RH

Lᵢ=Length of the hair in the straight configuration

The greater the relaxing efficiency (% RE), the straighter the hair after treatment. The results are shown in Table 2.

TABLE 2

| % Lysinate | pH of Final Mixture | % RE |
|---|---|---|
| 0 | 13.96 | 100 |
| 0.1 | 14.00 | 100 |
| 0.5 | 13.99 | 100 |
| 3.0 | 13.99 | 85 |
| 10 | 14.00 | 60 |

The results in Table 2 show that the relaxed hair, which had increased tensile strength when greater than 0.1% but less than 3.0% lysinate was present in the relaxing composition (as seen in Example 1), also maintained the desired relaxing efficiency (% RE) of a relaxing composition not comprising at least one organic nucleophile. This was evidenced by 100% RE for relaxing compositions further comprising at least one organic nucleophile wherein the at least one organic nucleophile was present in an amount greater than 0.1% and less than 3.0% lysinate (such as 0.1% and 0.5%).

### Example 3

### Tensile Strength of Hair Treated with a Relaxing Composition Further Comprising an Organic Nucleophile Other Than Lysinate

Following the procedure of Example 1, various amounts of isopropylamine and monoethanolamine (organic nucleophiles) were added to a commercially available lye-containing relaxing composition. These organic nucleophiles were added such that the final mixtures contained 2.50% by weight of available generated hydroxide ions and 0.404% of organic nucleophile as reported in Table 3. Results are shown in Table 3.

TABLE 3

| | % Nucleophile | |
|---|---|---|
| Nucleophile | in Final Mixture | % Decrease in W |
| None | 0 | 44.88 |
| Isopropylamine | 0.404 | 39.80 |
| Monoethanolamine | 0.404 | 40.45 |

The results in Table 3 show that the tensile strength of the treated hair increased (as reflected by the lower % Decrease in W) when the relaxing composition further comprised an organic nucleophile (isopropylamine or monoethanolamine).

Again following the procedure of Example 1, 10 grams of a 5.0% arginine solution were added to 90 grams of a commercially available lye-containing relaxing composition containing 2.90% by weight NaOH, such that the final mixture comprised 2.50% by weight of available generated hydroxide ions and the organic nucleophile concentrations as reported in Table 4. The results are shown in Table 4.

TABLE 4

| | % Nucleophile | |
|---|---|---|
| Nucleophile | in Final Mixture | % Decrease in W |
| None | 0 | 44.88 |
| Arginine | 0.5 | 31.59 |

The results in Table 4 show that the tensile strength of the treated hair increased (as reflected by the lower % Decrease in W) when the relaxing composition further comprised arginine.

### Example 4

### Tensile Strength of Hair Treated with a No-Lye Relaxing Composition Further Comprising an Organic Nucleophile

Following the procedure of Example 1, the % Decrease in W of hair treated with a commercially available no-lye relaxer was measured. Various amounts of the organic nucleophile sodium lysinate were added to an activator solution prior to mixing with a commercial calcium hydroxide cream base, such that the final mixture contained available generated hydroxide ions equivalent to 2.50% by weight NaOH and the nucleophile concentrations as reported in Table 5. The results are shown in Table 5.

TABLE 5

| % Lysinate | % Decrease in W |
|---|---|
| 0 | 79.57 |
| 0.5 | 78.75 |
| 1.0 | 74.74 |

The results in Table 5 show that the tensile strength of the treated hair increased (as reflected by the lower % Decrease in W) when the no-lye relaxing composition further comprised the organic nucleophile.

### Example 6

### Tensile Strength of Hair Treated with a Pretreatment Composition Comprising an Organic Nucleophile and a Lye-Containing Relaxer Composition

Naturally kinky hair was treated with two different pretreatment compositions each comprising an organic nucleophile. Thus, the hair was treated with either a lysine solution (1 g solution/g hair) or an arginine solution (1 g solution/g hair) for 10 20 minutes at room temperature, and was then allowed to dry. The treated hair was then relaxed with a commercially available lye-containing relaxing composition comprising 2.50% by weight NaOH for 20 minutes and rinsed. The concentration of the organic nucleophile in the pretreatment compositions varied as shown in Tables 6 (organic nucleophile=lysine) and 7 (organic nucleophile=arginine). The % Decrease in W was measured according to the procedure of Example 1. The results are shown in Tables 6 and 7.

TABLE 6

| % Lysine in | |
|---|---|
| Pretreatment | |
| Composition | % Decrease in W |
| 0 | 44.88 |
| 0.5 | 30.31 |
| 2.0 | 39.65 |
| 5.0 | 34.40 |
| 10.0 | 35.22 |

TABLE 7

| % Arginine in | |
|---|---|
| Pretreatment | |
| Composition | % Decrease in W |
| 0 | 44.88 |
| 0.5 | 39.48 |

The results in Tables 6 and 7 show that the tensile strength of hair that was relaxed with a lye-containing relaxing composition was increased when the hair was treated prior to relaxing with a pretreatment composition comprising at least one organic nucleophile (lysine in Table 6, or arginine in Table 7) (as reflected by the lower % Decrease in W). The results in Table 6 show that when the concentration of lysine in the pretreatment composition ranged from 0.5% to 10.0%, the tensile strength of the treated hair was increased.

### Example 6

### Tensile Strength of Hair Treated with a Pretreatment Composition Comprising an Organic Nucleophile and a No-Lye Relaxer Composition

Naturally kinky hair was treated with pretreatment compositions comprising various concentrations of an organic nucleophile (sodium lysinate). The hair was treated with a sodium lysinate solution (1 g solution/g hair) for 10 minutes at room temperature, and then allowed to dry. The treated hair was then relaxed with a commercially available no-lye relaxing composition for 20 minutes and rinsed. The concentration of the organic nucleophile in solution varied as shown in Table 8. The % Decrease in W was measured according to the procedures of Example 1. The results are shown in Table 8.

TABLE 8

| % Sodium Lysinate | |
|---|---|
| in Pretreatment Composition | % Decrease in W |
| 0 | 79.57 |
| 0.5 | 80.88 |
| 1.0 | 73.39 |

The results in Table 8 show that the tensile strength of hair that was relaxed with a no-lye relaxing composition was increased when the hair was treated prior to relaxing with a pretreatment composition comprising 1.0% sodium lysinate (as reflected by the lower % Decrease in W).

What is claimed is:

1. A composition for lanthionizing keratin fibers comprising at least one organic nucleophile and at least one hydroxide ion generator, wherein said at least one organic nucleophile is present in an amount effective to increase the tensile strength of said keratin fibers, said amount ranging from greater than 0. 1% but less than 3% by weight relative to the total weight of the composition, with the proviso that if said at least one organic nucleophile is chosen from cysteine and derivatives thereof, said at least one organic nucleophile is present in an amount greater than 1.5% but less than 3% by weight relative to the total weight of said composition.

2. A composition for lanthionizing keratin fibers according to claim 1, wherein said at least one organic nucleophile is generated by at least one organic nucleophile source.

3. A composition for lanthionizing keratin fibers according to claim 2, wherein said at least one organic nucleophile is generated in situ.

4. A composition for lanthionizing keratin fibers according to claim 1, wherein said at least one organic nucleophile is chosen from basic amino acids, amines, alcohols, and mercaptans.

5. A composition for lanthionizing keratin fibers according to claim 2, wherein said at least one organic nucleophile source is chosen from derivatives of basic amino acids, derivatives of amines, derivatives of alcohols, and derivatives of mercaptans.

6. A composition for lanthionizing keratin fibers according to claim 4, wherein said basic amino acids are chosen from lysine, arginine, and histidine.

7. A composition for lanthionizing keratin fibers according to claim 6, wherein said basic amino acids are chosen from lysine and arginine.

8. A composition for lanthionizing keratin fibers according to claim 7, wherein said basic amino acids are chosen from lysine and arginine in a non-ionic form, an ammonium form, and a carboxylate form.

9. A composition for lanthionizing keratin fibers according to claim 4, wherein said at least one organic nucleophile is chosen from amines of the following formula and salts thereof:
N(R)₃
wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

10. A composition for lanthionizing keratin fibers according to claim 9, wherein R comprises from 1 to 6 carbon atoms.

11. A composition for lanthionizing keratin fibers according to claim 10, wherein R comprises from 1 to 4 carbon atoms.

12. A composition for lanthionizing keratin fibers according to claim 9, wherein at least one R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

13. A composition for lanthionizing keratin fibers according to claim 12, wherein R comprises from 1 to 6 carbon atoms.

14. A composition for lanthionizing keratin fibers according to claim 13, wherein R comprises from 1 to 4 carbon atoms.

15. A composition for lanthionizing keratin fibers according to claim 9, wherein said salts are chosen from ammonium salts, alkali metal salts, alkaline earth metal salts, organic acid addition salts and inorganic acid addition salts.

16. A composition for lanthionizing keratin fibers according to claim 15, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

17. A composition for lanthionizing keratin fibers according to claim 4; wherein said amines are chosen from isopropylamine, monoethanolamine, and aminomethylpropanol.

18. A composition for lanthionizing keratin fibers according to claim 4, wherein said at least one organic nucleophile is chosen from alcohols of the following formula and salts thereof:
R—OH
wherein R is chosen from linear, branched, substituted, and unsubstituted C₁-C₁₀ alkyl groups, and linear, branched, substituted, and unsubstituted C₁-C₁₀ alkenyl groups.

19. A composition for lanthionizing keratin fibers according to claim 18, wherein R comprises from 1 to 6 carbon atoms.

20. A composition for lanthionizing keratin fibers according to claim 19, wherein R comprises from 1 to 4 carbon atoms.

21. A composition for lanthionizing keratin fibers according to claim 18, wherein R is substituted with at least one group chosen from —COOR′, —COON(R′)₂, —OH, —SH, —N(R′)₂ and salts of any of the foregoing, wherein each R′ is independently chosen from a hydrogen atom, linear, branched, substituted, and unsubstituted C₁-C₁₀ alkyl groups, and linear, branched, substituted, and unsubstituted C₁-C₁₀ alkenyl groups.

22. A composition for lanthionizing keratin fibers according to claim 21, wherein R comprises from 1 to 6 carbon atoms.

23. A composition for lanthionizing keratin fibers according to claim 22, wherein R comprises from 1 to 4 carbon atoms.

24. A composition for lanthionizing keratin fibers according to claim 18, wherein said salts are chosen from ammonium salts, alkali metal salts, alkaline earth metal salts, organic acid addition salts and inorganic acid addition salts.

25. A composition for lanthionizing keratin fibers according to claim 24, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

26. A composition for lanthionizing keratin fibers according to claim 4, wherein said at least one organic nucleophile is chosen from mercaptans of the following formula and salts thereof:
R—SH
wherein R is chosen from linear, branched, substituted, and unsubstituted C₁-C₁₀ alkyl groups, and linear, branched, substituted, and unsubstituted C₁-C₁₀ alkenyl groups.

27. A composition for lanthionizing keratin fibers according to claim 26, wherein R comprises from 1 to 6 carbon atoms.

28. A composition for lanthionizing keratin fibers according to claim 27, wherein R comprises from 1 to 4 carbon atoms.

29. A composition for lanthionizing keratin fibers according to claim 26, wherein R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C,₀ alkenyl groups.

30. A composition for lanthionizing keratin fibers according to claim 29, wherein R comprises from 1 to 6 carbon atoms.

31. A composition for lanthionizing keratin fibers according to claim 30, wherein R comprises from 1 to 4 carbon atoms.

32. A composition for lanthionizing keratin fibers according to claim 26, wherein said salts are chosen from ammonium salts, alkali metal salts, alkaline earth metal salts, organic acid addition salts, and inorganic acid addition salts.

33. A composition for lanthionizing keratin fibers according to claim 32, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

34. A composition for lanthionizing keratin fibers according to claim 1, further comprising at least one other constituent chosen from solvents; preservatives; perfumes; UV filters; active hair care agents; plasticizers; anionic, cationic, amphoteric, nonionic, and zwitterionic surfactants; hair conditioning agents; silicone fluids; fatty esters; fatty alcohol, fatty chain hydrocarbons; emollients; lubricants; penetrants; anionic, cationic, amphoteric, nonionic, and zwitterionic polymers; dyes; tints; bleaches; reducing agents; pH adjusting agents; sunscreens; thickening agents; and at least one agent chosen from chelating agents, sequestering agents and salts thereof.

35. A composition for lanthionizing keratin fibers according to claim 1, further comprising at least one additional nucleophile different from said at least one organic nucleophile.

36. A pretreatment composition for keratin fibers comprising at least one organic nucleophile, wherein said pretreatment composition is applied to said keratin fibers prior to applying a relaxing composition, and further wherein said at least one organic nucleophile is present in an amount effective to increase the tensile strength of said keratin fibers.

37. A pretreatment composition for lanthionizing keratin fibers according to claim 36, wherein said at least one organic nucleophile is generated by at least one organic nucleophile source.

38. A pretreatment composition for lanthionizing keratin fibers according to claim 36, wherein said at least one organic nucleophile is generated in situ.

39. A pretreatment composition for lanthionizing keratin fibers according to claim 36, wherein said at least one organic nucleophile is chosen from basic amino acids, amines, alcohols, and mercaptans.

40. A pretreatment composition for lanthionizing keratin fibers according to claim 37, wherein said at least one organic nucleophile source is chosen from derivatives of basic amino acids, derivatives of amines, derivatives of alcohols, and derivatives of mercaptans.

41. A pretreatment composition for lanthionizing keratin fibers according to claim 39, wherein said basic amino acids are chosen from iysine, arginine, and histidine.

42. A pretreatment composition for lanthionizing keratin fibers according to claim 41, wherein said basic amino acids are chosen from lysine and arginine.

43. A pretreatment composition for lanthionizing keratin fibers according to claim 42, wherein said basic amino acids are chosen from lysine and arginine in a non-ionic form, an ammonium form, and a carboxylate form.

44. A pretreatment composition for lanthionizing keratin fibers according to claim 39, wherein said at least one organic nucleophile is chosen from amines of the following formula and salts thereof:
N(R)₃
wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

45. A pretreatment composition for lanthionizing keratin fibers according to claim 44, wherein R comprises from 1 to 6 carbon atoms.

46. A pretreatment composition for lanthionizing keratin fibers according to claim 45, wherein R comprises from 1 to 4 carbon atoms.

47. A pretreatment composition for lanthionizing keratin fibers according to claim 44, wherein at least one R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

48. A pretreatment composition for lanthionizing keratin fibers according to claim 47, wherein R comprises from 1 to 6 carbon atoms.

49. A pretreatment composition for lanthionizing keratin fibers according to claim 48, wherein R comprises from 1 to 4 carbon atoms.

50. A pretreatment composition for lanthionizing keratin fibers according to claim 44, wherein said salts are chosen from ammonium salts, alkali metal salts, alkaline earth metal salts, organic acid addition salts and inorganic acid addition salts.

51. A pretreatment composition for lanthionizing keratin fibers according to claim 50, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

52. A pretreatment composition for lanthionizing keratin fibers according to claim 39, wherein said amines are chosen from isopropylamine, monoethanolamine, and aminomethylpropanol.

53. A pretreatment composition for lanthionizing keratin fibers according to claim 39, wherein said at least one organic nucleophile is chosen from alcohols of the following formula and salts thereof:
R—OH
wherein R is chosen from linear, branched, substituted, and unsubstituted C₁-C₁₀ alkyl groups, and linear, branched, substituted, and unsubstituted C₁-C₁₀ alkenyl groups.

54. A pretreatment composition for lanthionizing keratin fibers according to claim 53, wherein R comprises from 1 to 6 carbon atoms.

55. A pretreatment composition for lanthionizing keratin fibers according to claim 54, wherein R comprises from 1 to 4 carbon atoms.

56. A pretreatment composition for lanthionizing keratin fibers according to claim 53, wherein R is substituted with at least one group chosen from —COOR′, —COON(R′)₂, —OH, —SH, —N(R′)₂ and salts of any of the foregoing, wherein each R′ is independently chosen from a hydrogen atom, linear, branched, substituted, and unsubstituted C₁-C₁₀ alkyl groups, and linear, branched, substituted, and unsubstituted C₁-C₁₀ alkenyl groups.

57. A pretreatment composition for lanthionizing keratin fibers according to claim 56, wherein R comprises from 1 to 6 carbon atoms.

58. A pretreatment composition for lanthionizing keratin fibers according to claim 57, wherein R comprises from 1 to 4 carbon atoms.

59. A pretreatment composition for lanthionizing keratin fibers according to claim 53, wherein said salts are chosen from ammonium salts, alkali metal salts, alkaline earth metal salts, organic acid addition salts and inorganic acid addition salts.

60. A pretreatment composition for lanthionizing keratin fibers according to claim 59, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

61. A pretreatment composition for lanthionizing keratin fibers according to claim 39, wherein said at least one organic nucleophile is chosen from mercaptans of the following formula and salts thereof:
R—SH
wherein R is chosen from linear, branched, substituted, and unsubstituted C₁-C₁₀ alkyl groups, and linear, branched, substituted, and unsubstituted C₁-C₁₀ alkenyl groups.

62. A pretreatment composition for lanthionizing keratin fibers according to claim 61, wherein R comprises from 1 to 6 carbon atoms.

63. A pretreatment composition for lanthionizing keratin fibers according to claim 62, wherein R comprises from 1 to 4 carbon atoms.

64. A pretreatment composition for lanthionizing keratin fibers according to claim 61, wherein R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

65. A pretreatment composition for lanthionizing keratin fibers according to claim 64, wherein R comprises from 1 to 6 carbon atoms.

66. A pretreatment composition for lanthionizing keratin fibers according to claim 65, wherein R comprises from 1 to 4 carbon atoms.

67. A pretreatment composition for lanthionizing keratin fibers according to claim 61, wherein said salts are chosen from ammonium salts, alkali metal salts, alkaline earth metal salts, organic acid addition salts and inorganic acid addition salts.

68. A pretreatment composition for lanthionizing keratin fibers according to claim 67, wherein said salts are chosen from sodium salts, potassium salts, calcium salts, salts derived from hydrochloric acid, salts derived from sulphuric acid, salts de rived from phosphoric acid, salts derived from acetic acid, salts derived from citric acid, and salts derived from tartaric acid.

69. A pretreatment composition for lanthionizing keratin fibers according to claim 36, further comprising at least one other constituent chosen from solvents; preservatives; perfumes; UV filters; active hair care agents; plasticizers; anionic, cationic, amphoteric, nonionic, and zwitterionic surfactants; hair conditioning agents; silicone fluids; fatty esters; fatty alcohol, fatty chain hydrocarbons; emollients; lubricants; penetrants; anionic, cationic, amphoteric, nonionic, and zwitterionic polymers; dyes; tints; bleaches; reducing agents; pH adjusting agents; sunscreens; thickening agents; and at least one agent chosen from chelating agents, sequestering agents and salts thereof.

70. A pretreatment composition for lanthionizing keratin fibers according to claim 36, further comprising at least one additional nucleophile different from said at least one nucleophile.

71. A pretreatment composition for lanthionizing keratin fibers according to claim 36, wherein said at least one organic nucleophile is present in an amount ranging up to 100% by weight relative to the total weight of the pretreatment composition.

72. A pretreatment composition for lanthionizing keratin fibers according to claim 71, wherein said at least one organic nucleophile is present in an amount ranging from 0.001% to 10.0% by weight relative to the total weight of the pretreatment composition.

73. A pretreatment composition for lanthionizing keratin fibers according to claim 72, wherein said at least one organic nucleophile is present in an amount ranging from 0.01% to 2.0% by weight relative to the total weight of the pretreatment composition.

74. A pretreatment composition for lanthionizing keratin fibers according to claim 73, wherein said at least one organic nucleophile is present in an amount of 0.2% by weight relative to the total weight of the pretreatment composition.

75. A method for lanthionizing keratin fibers to achieve relaxation of said keratin fibers, comprising:
applying to said keratin fibers a pretreatment composition comprising at least one organic nucleophile, wherein said at least one organic nucleophile is present in an amount effective to increase the tensile strength of said keratin fibers;
applying a relaxing composition to said pre-treated keratin fibers for a sufficient period of time to lanthionize said keratin fibers; and
terminating said lanthionization when a desired level of relaxation of said keratin fibers has been reached.

76. A method for lanthionizing keratin fibers to achieve relaxation of said keratin fibers according to claim 75, wherein said at least one organic nucleophile is generated by at least one organic nucleophile source.

77. A method for lanthionizing keratin fibers to achieve relaxation of said keratin fibers according to claim 75, wherein said at least one organic nucleophile is generated in situ.

78. A method for lanthionizing keratin fibers to achieve relaxation of said keratin fibers according to claim 75, wherein said relaxing composition comprises at least one hydroxide ion generator which generates hydroxide ions in situ.

79. A method for lanthionizing keratin fibers to achieve relaxation of said keratin fibers according to claim 75, wherein said at least one organic nucleophile is chosen from basic amino acids, amines, alcohols, and mercaptans.

80. A method for lanthionizing keratin fibers to achieve relaxation of said keratin fibers according to claim 76, wherein said at least one organic nucleophile source is chosen from derivatives of basic amino acids, derivatives of amines, derivatives of alcohols, and derivatives of mercaptans.

81. A method for lanthionizing keratin fibers to achieve relaxation of said keratin fibers according to claim 79, wherein said basic amino acids are chosen from lysine, arginine, and histidine.

82. A method for lanthionizing keratin fibers to achieve relaxation of said keratin fibers according to claim 81, wherein said basic amino acids are chosen from lysine and arginine.

83. A method for lanthionizing keratin fibers according to claim 82, wherein said basic amino acids are chosen from lysine and arginine in a non-ionic form, an ammonium form, and a carboxylate form.

84. A method for lanthionizing keratin fibers to achieve relaxation of said keratin fibers according to claim 79, wherein said at least one organic nucleophile is chosen from amines of the following formula and salts thereof:
N(R)₃
wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

85. A method for lanthionizing keratin fibers to achieve relaxation of said keratin fibers according to claim 84, wherein R comprises from 1 to 6 carbon atoms.

86. A method for lanthionizing keratin fibers to achieve relaxation of said keratin fibers according to claim 85, wherein R comprises from 1 to 4 carbon atoms.

87. A method for lanthionizing keratin fibers to achieve relaxation of said keratin fibers according to claim 84, wherein at least one R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

88. A method for lanthionizing keratin fibers to achieve relaxation of said keratin fibers according to claim 87, wherein R comprises from 1 to 6 carbon atoms.

89. A method for lanthionizing keratin fibers to achieve relaxation of said keratin fibers according to claim 88, wherein R comprises from 1 to 4 carbon atoms.

90. A method for lanthionizing keratin fibers to achieve relaxation of said keratin fibers according to claim 84, wherein said salts are chosen from ammonium salts, alkali metal salts, alkaline earth metal salts, organic acid addition salts and inorganic acid addition salts.

91. A method for lanthionizing keratin fibers to achieve relaxation of said keratin fibers according to claim 90, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

92. A method for lanthionizing keratin fibers to achieve relaxation of said keratin fibers according to claim 79, wherein said amines are chosen from isopropylamine, monoethanolamine, and aminomethylpropanol.

93. A method for lanthionizing keratin fibers to achieve relaxation of said keratin fibers according to claim 75, wherein said at least one organic nucleophile is chosen from alcohols of the following formula and salts thereof:
R—OH
wherein R is chosen from linear, branched, substituted, and unsubstituted C₁-C₁₀ alkyl groups, and linear, branched, substituted, and unsubstituted C₁-C₁₀ alkenyl groups.

94. A method for lanthionizing keratin fibers to achieve relaxation of said keratin fibers according to claim 93, wherein R comprises from 1 to 6 carbon atoms.

95. A method for lanthionizing keratin fibers to achieve relaxation of said keratin fibers according to claim 94, wherein R comprises from 1 to 4 carbon atoms.

96. A method for lanthionizing keratin fibers to achieve relaxation of said keratin fibers according to claim 93, wherein R is substituted with at least one group chosen from —COOR′, —COON(R′)₂, —OH, —SH, —N(R′)₂ and salts of any of the foregoing, wherein each R′ is independently chosen from a hydrogen atom, linear, branched, substituted, and unsubstituted C₁-C₁₀ alkyl groups, and linear, branched, substituted, and unsubstituted C₁-C₁₀ alkenyl groups.

97. A method for lanthionizing keratin fibers to achieve relaxation of said keratin fibers according to claim 96, wherein R comprises from 1 to 6 carbon atoms.

98. A method for lanthionizing keratin fibers to achieve relaxation of said keratin fibers according to claim 97, wherein R comprises from 1 to 4 carbon atoms.

99. A method for lanthionizing keratin fibers to achieve relaxation of said keratin fibers according to claim 96, wherein said salts are chosen from ammonium salts, alkali metal salts, alkaline earth metal salts, organic acid addition salts and inorganic acid addition salts.

100. A method for lanthionizing keratin fibers to achieve relaxation of said keratin fibers according to claim 99, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

101. A method for lanthionizing keratin fibers to achieve relaxation of said keratin fibers according to claim 75, wherein said at least one organic nucleophile is chosen from mercaptans of the following formula and salts thereof:
R—SH
wherein R is chosen from linear, branched, substituted, and unsubstituted C₁-C₁₀ alkyl groups, and linear, branched, substituted, and unsubstituted C₁-C₁₀ alkenyl groups.

102. A method for lanthionizing keratin fibers to achieve relaxation of said keratin fibers according to claim 101, wherein R comprises from 1 to 6 carbon atoms.

103. A method for lanthionizing keratin fibers to achieve relaxation of said keratin fibers according to claim 102, wherein R comprises from 1 to 4 carbon atoms.

104. A method for lanthionizing keratin fibers to achieve relaxation of said keratin fibers according to claim 101, wherein R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

105. A method for lanthionizing keratin fibers to achieve relaxation of said keratin fibers according to claim 104, wherein R comprises from 1 to 6 carbon atoms.

106. A method for lanthionizing keratin fibers to achieve relaxation of said keratin fibers according to claim 105, wherein R comprises from 1 to 4 carbon atoms.

107. A method for lanthionizing keratin fibers to achieve relaxation of said keratin fibers according to claim 101, wherein said salts are chosen from ammonium salts, alkali metal salts, alkaline earth metal salts, organic acid addition salts and inorganic acid addition salts.

108. A method for lanthionizing keratin fibers to achieve relaxation of said keratin fibers according to claim 107, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

109. A method for lanthionizing keratin fibers to achieve relaxation of said keratin fibers according to claim 75, wherein at least one of said pretreatment composition and said relaxing composition further comprises at least one other constituent chosen from solvents; preservatives; perfumes; UV filters; active hair care agents; plasticizers; anionic, cationic, amphoteric, nonionic, and zwitterionic surfactants; hair conditioning agents; silicone fluids; fatty esters; fatty alcohol, fatty chain hydrocarbons; emollients; lubricants; penetrants; anionic, cationic, amphoteric, nonionic, and zwitterionic polymers; dyes; tints; bleaches; reducing agents; pH adjusting agents; sunscreens; thickening agents; and at least one agent chosen from chelating agents, sequestering agents and salts thereof.

110. A method for lanthionizing keratin fibers to achieve relaxation of said keratin fibers according to claim 75, wherein at least one of said pretreatment composition and said relaxing composition further comprises at least one additional nucleophile different from said at least one nucleophile.

111. A method for lanthionizing keratin fibers to achieve relaxation of said keratin fibers according to claim 75, wherein said at least one organic nucleophile is present in an amount ranging up to 100% by weight relative to the total weight of the pretreatment composition.

112. A method for lanthionizing keratin fibers to achieve relaxation of said keratin fibers according to claim 111, wherein said at least one organic nucleophile is present in an amount ranging from 0.001% to 10.0% by weight relative to the total weight of the pretreatment composition.

113. A method for lanthionizing keratin fibers to achieve relaxation of said keratin fibers according to claim 112, wherein said at least one organic nucleophile is present in an amount ranging from 0.01% to 2.0% by weight relative to the total weight of the pretreatment composition.

114. A method for lanthionizing keratin fibers to achieve relaxation of said keratin fibers according to claim 113, wherein said at least one organic nucleophile is present in an amount of 2.0% by weight relative to the total weight of the pretreatment composition.

115. A method for lanthionizing keratin fibers to achieve relaxation of said keratin fibers, comprising:
applying a relaxing composition to keratin fibers for a sufficient period of time to lanthionize said keratin fibers, wherein said relaxing composition further comprises at least one organic nucleophile, and further wherein said at least one organic nucleophile is present in an amount effective to increase the tensile strength of said keratin fibers ranging from greater than 0.1% but less than 3% by weight relative to the total weight of the relaxing composition; and
terminating said lanthionization when a desired level of relaxation of said keratin fibers has been reached.

116. A method for lanthionizing keratin fibers to achieve relaxation of said keratin fibers according to claim 115, wherein said at least one organic nucleophile is generated by at least one organic nucleophile source.

117. A method for lanthionizing keratin fibers to achieve relaxation of said keratin fibers according to claim 115, wherein said at least one organic nucleophile is generated in situ.

118. A method for lanthionizing keratin fibers to achieve relaxation of said keratin fibers according to claim 115, wherein said relaxing composition comprises at least one hydroxide ion generator which generates hydroxide ions in situ.

119. A method for lanthionizing keratin fibers to achieve relaxation of said keratin fibers according to claim 115, wherein said at least one organic nucleophile is chosen from basic amino acids, amines, alcohols, and mercaptans.

120. A method for lanthionizing keratin fibers to achieve relaxation of said keratin fibers according to claim 116, wherein said at least one organic nucleophile source is chosen from derivatives of basic amino acids, derivatives of amines, derivatives of alcohols, and derivatives of mercaptans.

121. A method for lanthionizing keratin fibers to achieve relaxation of said keratin fibers according to claim 117, wherein said basic amino acids are chosen from lysine, arginine, and histidine.

122. A method for lanthionizing keratin fibers to achieve relaxation of said keratin fibers according to claim 121, wherein said basic amino acids are chosen from lysine and arginine.

123. A method for lanthionizing keratin fibers according to claim 122, wherein said basic amino acids are chosen from lysine and arginine in a non-ionic form, an ammonium form, and a carboxylate form.

124. A method for lanthionizing keratin fibers to achieve relaxation of said keratin fibers according to claim 119, wherein said at least one organic nucleophile is chosen from amines of the following formula and salts thereof:
N(R)₃
wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

125. A method for lanthionizing keratin fibers to achieve relaxation of said keratin fibers according to claim 124, wherein R comprises from 1 to 6 carbon atoms.

126. A method for lanthionizing keratin fibers to achieve relaxation of said keratin fibers according to claim 125, wherein R comprises from 1 to 4 carbon atoms.

127. A method for lanthionizing keratin fibers to achieve relaxation of said keratin fibers according to claim 124, wherein at least one R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

128. A method for lanthionizing keratin fibers to achieve relaxation of said keratin fibers according to claim 127, wherein R comprises from 1 to 6 carbon atoms.

129. A method for lanthionizing keratin fibers to achieve relaxation of said keratin fibers according to claim 128, wherein R comprises from 1 to 4 carbon atoms.

130. A method for lanthionizing keratin fibers to achieve relaxation of said keratin fibers according to claim 124, wherein said salts are chosen from ammonium salts, alkali metal salts, alkaline earth metal salts, organic acid addition salts and inorganic acid addition salts.

131. A method for lanthionizing keratin fibers to achieve relaxation of said keratin fibers according to claim 130, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

132. A method for lanthionizing keratin fibers to achieve relaxation of said keratin fibers according to claim 124, wherein said amines are chosen from isopropylamine, monoethanolamine, and aminomethylpropanol.

133. A method for lanthionizing keratin fibers to achieve relaxation of said keratin fibers according to claim 119, wherein said at least one organic nucleophile is chosen from alcohols of the following formula and salts thereof:
R—OH
wherein R is chosen from linear, branched, substituted, and unsubstituted C₁-C₁₀ alkyl groups, and linear, branched, substituted, and unsubstituted C1-C10 alkenyl groups.

134. A method for lanthionizing keratin fibers to achieve relaxation of said keratin fibers according to claim 133, wherein R comprises from 1 to 6 carbon atoms.

135. A method for lanthionizing keratin fibers to achieve relaxation of said keratin fibers according to claim 134, wherein R comprises from 1 to 4 carbon atoms.

136. A method for lanthionizing keratin fibers to achieve relaxation of said keratin fibers according to claim 133, wherein R is substituted with at least one group chosen from —COOR′, —COON(R′)₂, —OH, —SH, —N(R′)₂ and salts of any of the foregoing, wherein each R′ is independently chosen from a hydrogen atom, linear, branched, substituted, and unsubstituted C₁-C₁₀ alkyl groups, and linear, branched, substituted, and unsubstituted C₁-C₁₀ alkenyl groups.

137. A method for lanthionizing keratin fibers to achieve relaxation of said keratin fibers according to claim 136, wherein R comprises from 1 to 6 carbon atoms.

138. A method for lanthionizing keratin fibers to achieve relaxation of said keratin fibers according to claim 137, wherein R comprises from 1 to 4 carbon atoms.

139. A method for lanthionizing keratin fibers to achieve relaxation of said keratin fibers according to claim 133, wherein said salts are chosen from ammonium salts, alkali metal salts, alkaline earth metal salts, organic acid addition salts and inorganic acid addition salts.

140. A method for lanthionizing keratin fibers to achieve relaxation of said keratin fibers according to claim 139, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

141. A method for lanthionizing keratin fibers to achieve relaxation of said keratin fibers according to claim 119, wherein said at least one organic nucleophile is chosen from mercaptans of the following formula and salts thereof:
R—SH
wherein R is chosen from linear, branched, substituted, and unsubstituted C₁-C₁₀ alkyl groups, and linear, branched, substituted, and unsubstituted C₁-C₁₀ alkenyl groups.

142. A method for lanthionizing keratin fibers to achieve relaxation of said keratin fibers according to claim 141, wherein R comprises from 1 to 6 carbon atoms.

143. A method for lanthionizing keratin fibers to achieve relaxation of said keratin fibers according to claim 142, wherein R comprises from 1 to 4 carbon atoms.

144. A method for lanthionizing keratin fibers to achieve relaxation of said keratin fibers according to claim 141, wherein R is substituted with at least one group chosen from —COOR, —COON(R)₂, —OH, —SH, —N(R)₂ and salts of any of the foregoing, wherein each R is independently chosen from a hydrogen atom, linear, branched, substituted, and unsubstituted C₁-C₁₀ alkyl groups, and linear, branched, substituted, and unsubstituted C₁-C₁₀ alkenyl groups.

145. A method for lanthionizing keratin fibers to achieve relaxation of said keratin fibers according to claim 144, wherein R comprises from 1 to 6 carbon atoms.

146. A method for lanthionizing keratin fibers to achieve relaxation of said keratin fibers according to claim 145, wherein R comprises from 1 to 4 carbon atoms.

147. A method for lanthionizing keratin fibers to achieve relaxation of said keratin fibers according to claim 141, wherein said salts are chosen from ammonium salts, alkali metal salts, alkaline earth metal salts, organic acid addition salts, and inorganic acid addition salts.

148. A method for lanthionizing keratin fibers to achieve relaxation of said keratin fibers according to claim 147, wherein said salts are chosen from sodium salts, potassium salts, calcium salts, salts derived from hydrochloric acid, salts derived from sulphuric acid, salts derived from phosphoric acid, salts derived from acetic acid, salts derived from citric acid, and salts derived from tartaric acid.

149. A method for lanthionizing keratin fibers to achieve relaxation of said keratin fibers according to claim 115, wherein said relaxing composition further comprises at least one other constituent chosen from solvents; preservatives; perfumes; UV filters; active hair care agents; plasticizers; anionic, cationic, amphoteric, nonionic, and zwitterionic surfactants; hair conditioning agents; silicone fluids; fatty esters; fatty alcohol, fatty chain hydrocarbons; emollients; lubricants; penetrants; anionic, cationic, amphoteric, nonionic, and zwitterionic polymers; dyes; tints; bleaches; reducing agents; pH adjusting agents; sunscreens; and thickening agents.

150. A method for lanthionizing keratin fibers to achieve relaxation of said keratin fibers according to claim 115, wherein said relaxing composition further comprises at least one additional nucleophile different from said at least one organic nucleophile.

151. A multicomponent kit for lanthionizing keratin fibers comprising at least two components, which are separate from each other,
wherein a first component comprises at least one organic nucleophile, and
wherein a second component comprises at least one hydroxide ion generator.

152. A multicomponent kit for lanthionizing keratin fibers according to claim 151, wherein said at least one organic nucleophile is generated by at least one organic nucleophile source.

153. A multicomponent kit for lanthionizing keratin fibers according to claim 151, wherein said first component is to be applied to said keratin fibers before said second component.

154. A multicomponent kit for lanthionizing keratin fibers according to claim 151, wherein said first component is combined with said second component prior to application to said keratin fibers.

155. A multicomponent kit for lanthionizing keratin fibers according to claim 151, wherein at least one of said first component and said second component is in a form chosen from an emulsion, a solution, a suspension, a gel, a cream, and a paste.

156. A multicomponent kit for lanthionizing keratin fibers according to claim 151, wherein said keratin fibers are hair.

157. A composition according to claim 1, wherein said keratin fibers are hair.

158. A pretreatment composition for lanthionizing keratin fibers according to claim 36, wherein said keratin fibers are hair.

159. A method for lanthionizing keratin fibers according to claim 75, wherein said keratin fibers are hair.

160. A method for lanthionizing keratin fibers to achieve relaxation of said keratin fibers according to claim 115, wherein said keratin fibers are hair.
